(19) United States
(12) Patent Application Publication
(10) Pub. No.: US 2020/0129602 A1
(43) Pub. Date: Apr. 30, 2020

(54) Vaccine for Malignant Tumor Treatment

(71) Applicant: Intellexon GmbH, Pöcking (DE)

(72) Inventor: Wolfgang Würfel, Sixtnitgern (DE)

(21) Appl. No.: 16/621,188

(22) PCT Filed: Jun. 19, 2018
(86) PCT No.: PCT/EP18/66211

§ 371 Date: Dec. 10, 2019

(30) Foreign Application Priority Data
Jun. 20, 2017 (DE) 10 2017 005 815.6

(51) Int. Cl. A61K 39/00; C40B 40/14; G01N 33/569

(52) U.S. Cl. CPC A61K 39/0011; G01N 2800/7028; G01N 33/56977; C40B 40/14

(57) ABSTRACT

Disclosed are methods for preparing a medicament for treating a malignant tumor, along with cell membranes prepared by such methods and the use thereof, in which the individual communication structure between the malignant tumor and the immune system is ascertained based upon a tissue sample containing cells of the malignant tumor by determining a malignant tumor-specific expression pattern of histocompatibility antigens (Human Leucocyte Antigen, HLA) on said tissue sample, masking or removing at least a part of the expression pattern present on the cells of the tissue sample that is capable of exerting an inhibitory effect on immunocompetent cells, and preparing an individual vaccine for eliciting a specific immunological response by lysing those cells on which a part of the expression pattern has been masked or removed.

### FIELD OF THE INVENTION

The present invention relates to the area of providing vaccines for the treatment of malignant tumors.

### BACKGROUND OF THE INVENTION

In addition to classic therapies for the treatment of malignant tumor diseases, e.g., resection, chemotherapy, and radiation therapy, the use of active or passive immunotherapies based on the administration of vaccines for initiating an immunological response or the administration of antibodies (antibody fragments) for binding to the malignant tumor is increasing. Medicaments for treating malignant tumors should be highly selective and should not allow any resistances to be produced.

As the goal for an immunological therapy, neoantigens may be used, for example. Neoantigens are proteins or protein-like molecules that are antigenic in nature (and thus elicit a response in immunocompetent cells) and are based on new mutations in the genome occurring as part of malignant degeneration. Examples of these include neoantigens that elicit a response of T cells, in particular a response of CD8+ T cells in the case of neoantigens presented by MHC-I (Major Histocompatibility Complex, Class I), or CD4+ T cells in the case of neoantigens presented by MHC II (Major Histocompatibility Complex, Class II).

Based on a detection of such new mutations in an individual malignant tumor by means of RNA analysis, mass spectrometry, or sequencing, for example, an individual vaccine can be developed and produced, e g. by in-vitro culture with dendritic cells. However, this detection can be cumbersome and prone to error, in particular due to the uncertainty as to which new mutations will be expressed as neoantigens, and due to neoantigens that are derived from oncogenes or splicing variants and are not based on new mutations. The high individuality and in some cases chaotic cell organization leads to differences, even within a tumor disease, e.g. between neoantigens of metastases and neoantigens of the primary tumor.

Nevertheless, tumor diseases have mechanisms for evading an immunological response. One such so-called escape mechanism is based on the MHC (Major Histocompatibility Complex), in particular with its histocompatibility antigen groups (Human Leucocyte Antigen), which serves for the cellular dialog in humans. In the literature, the abbreviation HLA may be used to designate coding genes or proteins expressed by them. The concept of HLA groups as used in the following refers to the surface proteins expressed by the genes on the cell surface.

In general, HLA groups can be divided into the following four classes:

(i) HLA groups A, B, and C (MHC-I), which essentially identify all adult and somatic cells;
(ii) HLA groups D (DRB, DQB, etc.; MHC-II), which play an important part in the presentation of antigens for immunocompetent cells;
(iii) HLA groups E, F, and G, which identify embryonic cells, in particular on the so-called invasion front;
(iv) HLA groups H, et seq., the so-called pseudogenes.

Malignant tumor cells can express characteristic “embryonic” HLA groups (i.e., HLA-E, HLA-F and/or HLA-G) on their surface “Embryonic” HLA groups can contribute to malignant tumor cells evading attack by the non-specific and/or specific immunological defense of the organism itself. As a result of the expression of these characteristic HLA groups on the surface of the cells, the latter are rendered capable of activating corresponding receptors for immunocompetent cells. In general, these are receptors that, after activation, inhibit the functioning of these immunocompetent cells, for example, the killer immunoglobulin-like receptors (KIR) on the natural killer cells or the leukocyte immunoglobulin-like receptors (LILR) on the lymphocytes.

Therefore, in particular the antigens HLA-E, F and G on the embryonic cells (especially on placental and trophoblastic cells) prevent the immune system of the mother from attacking the cells. In this way, embryos can evade the immunological response. This escape mechanism constitutes the backbone of the immunological control of pregnancy. A rejection response does not take place, and the genetically semi-foreign (father's foreign part 50%) or foreign embryo (in the case of single-cell donors or embryo donors or surrogate motherhood, 100%) can be carried to full term.

Malignant tumors of very different tissues are able to make use of this embryonic escape mechanism to suppress or diminish the immunological defense. It also enables them to counteract some therapeutic strategies, i.e., to inhibit strategies that are based on an attack. For this reason, it can be advantageous to take the escape mechanism into account and to incorporate the immune system in treating the malignant tumor.

Given this background, the invention has the object of providing methods for preparing medicaments, cell membranes for treating malignant tumors, and the use of such cell membranes.

### SUMMARY OF THE INVENTION

This object is attained by methods, cell membranes, and the use thereof according to the independent claims. The dependent claims describe preferred embodiments.

A method according to the invention for preparing a medicament for treating a malignant tumor comprises ascertaining the individual communication structure between the malignant tumor and the immune system and preparing an individual vaccine to elicit a specific immunological response.

To ascertain the individual communication structure between the malignant tumor and the immune system, a malignant tumor-specific expression pattern of histocompatibility antigens (Human Leucocyte Antigen, HLA) is determined on a tissue sample containing cells of the malignant tumor.

Even tumors or metastases that are classified as histopathologically identical can have expression patterns that are different inter-individually or intra-individually from one location to another location. Therapies, e.g., the administration of chemical therapeutic agents or hormonal antagonists, can further affect the expression patterns. A determination of the individual expression patterns addresses these differences.

The term malignant tumor cell also covers metastatic cells of the primary malignant tumor. The method according to the invention is preferably carried out for several, especially preferably for all metastases individually, in order to address any individual differences of the metastases, in particular their individual expression patterns of histocompatibility antigens.

To prepare an individual vaccine designed to elicit a specific immunological response, at least one part of the expression pattern present on the cells of the tissue sample that is capable of exerting an inhibitory effect on immunocompetent cells is masked or removed. By masking, i.e. blocking, or removing histocompatibility antigens, and thereby preventing the binding of immune system-side receptors to these HLA groups, their inhibitory effect on the immune system can be prevented.

Furthermore, those cells on which a part of the expression pattern has been masked or removed are lysed to obtain cell membranes or fragments of cell membranes for injection. At the same time, due to this lysis the destroyed malignant tumor cell no longer poses a danger.

In embodiments according to Claim 2, the histocompatibility antigens for which the expression pattern is determined comprise “embryonic” HLA groups, in particular HLA-E, F, and/or G.

In embodiments according to Claim 3, the part of the expression pattern to be masked or removed comprises the embryonic HLA groups, in particular HLA-E, F, and/or G.

In embodiments according to Claim 4, the at least one part of the expression pattern is masked by means of antibodies. Antibody masking can prevent the masked histocompatibility antigens from binding to inhibitory receptors of immunocompetent cells, thus disrupting the escape mechanism of the malignant tumor. Examples of such antibodies include anti-HLA-E antibodies, anti-HLA-F antibodies, and anti-HLA-G antibodies. Alternatively, combined or multivalent antibodies may be used.

In embodiments according to Claim 5, the at least one part of the expression pattern is removed by means of gene manipulation techniques. Removal by gene manipulation techniques can prevent binding of the removed histocompatibility antigens to inhibitory receptors of immunocompetent cells, thus disrupting the escape mechanism of the malignant tumor.

One example of such gene manipulation techniques is Crispr-CAS, in which the genes or DNA segments that code for immunoinhibitory histocompatibility antigens are excised, so that the cells of the tissue sample can no longer express these HLA groups. Other examples include techniques based on zinc finger nucleases, transcription activator-like effector nucleases (TALEN), or modified homing endonucleases.

In embodiments according to Claim 6, the cells are lysed by means of mechanical or biochemical cell disruption methods, in particular by means of hypotonic lysis. For example, the cells may be ruptured in a hypo-osmolar solution. In this way, cell membranes or cell membrane fragments with high antigenic effect can be obtained, in particular for injection in the form of a vaccine.

In embodiments according to Claim 7, the cell complex of the tissue sample is dissociated to obtain a single cell suspension. Dissociation may be performed enzymatically using trypsin or collagenase, for example.

The invention further provides cell membranes prepared by a method according to the invention.

In particular, the cell membranes may have an expression pattern of histocompatibility antigens, from which the part that is capable of inhibiting an inhibitory response of the immune system has been masked or removed. The prepared cell membranes are suitable for use as a vaccine for treating a malignant tumor by the specific activation of the immune system.

Additionally, the invention provides for the use of cell membranes according to the invention as a medicament for treating a malignant tumor. In particular, the cell membranes may be used in vivo as a vaccine for the specific activation of the immune system.

In some embodiments, the cell membranes can be used to “train” immunocompetent cells, in particular T cells, in vitro, i.e. to activate on the neoantigens, and to then reinject the trained or activated immunocompetent cells into the organism. For in vitro activation, immunocompetent cells may be removed, exposed to the cell membranes or the vaccine, and returned by transfusion after activation.

Embodiments according to Claim 10 comprise the use of the cell membranes as a vaccine containing the cell membranes or at least fragments of the cell membranes for the organism from which the tissue sample was taken, to elicit a specific activation or response of the immune system. In particular, the use may comprise an injection of the vaccine.

In some embodiments, usage of the cell membranes may take place locally or systemically. Local usage comprises, for example, injection into the malignant tumor or into its vicinity. Systemic usage comprises, for example, administration in one of the following ways: orally, nasally, sublingually, rectally, subcutaneously, intravenously, percutaneously, etc.

In some embodiments, the use may further comprise the use of checkpoint inhibitors and/or traditional adjuvants, such as bacillus Calmette Guerin (BCG), Freud's adjuvant, or aluminum hydroxide, to enhance the immunological response.

### BRIEF DESCRIPTION OF THE DRAWINGS

The attached drawings are referred to in the following description of exemplary embodiments:

FIG. 1 shows a flow chart of a method for preparing a medicament according to one exemplary embodiment

FIG. 2 shows a schematic view of a procedure for carrying out a method according to one embodiment

FIG. 3 shows a schematic view of a procedure for carrying out a method according to another embodiment

FIG. 4 shows a schematic view of a cell membrane according to one embodiment

FIG. 5 shows a use of a cell membrane according to one embodiment

### DESCRIPTION OF PREFERRED EXEMPLARY EMBODIMENTS

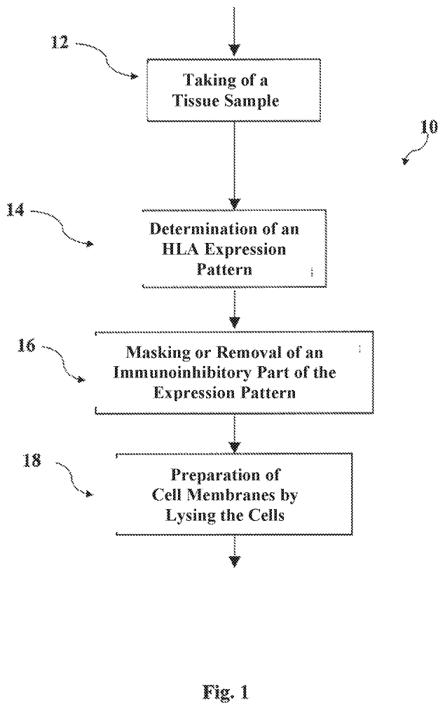

FIG. 1 shows a flow chart of a method 10 for preparing a medicament for treating a malignant tumor. The method 10 is preceded by the step of taking 12 a tissue sample. The method 10 comprises the steps of determining 14 an expression pattern, masking or removing 16 an immunoinhibitory part of the expression pattern, and preparing 18 cell membranes by lysing the cells.

In taking 12 a tissue sample, at least cells of the malignant tumor to be treated are extracted. The tissue sample may be obtained by surgery or biopsy, for example.

In determining 14 an expression pattern, a malignant tumor-specific expression pattern of histocompatibility antigens on the tissue sample is determined. The determination of the expression pattern may be limited to the known histocompatibility antigens (or some of them), i.e. it may comprise only a limited number of predefined proteins. The determination of the expression pattern can thereby be made more specific and less cumbersome than the determination of neoantigens whose number and composition are not limited or known a priori.

Determining the expression pattern preferably comprises the quantitative determination of an expression level. Expression patterns or expression levels may be determined by known methods such as RNA sequencing, DNA microarrays, quantitative PCR (polymerase chain reaction), expression profiling, SAGE (serial analysis of gene expression), etc., for example.

In the masking or removal 16 of an immunoinhibitory part of the expression pattern, at least a part of the expression pattern, present on the cells of the tissue sample, that is capable of exerting an inhibitory effect on immunocompetent cells is masked or removed. In some embodiments, in addition to the immunoinhibitory part of the expression pattern, other parts of the expression pattern may also be masked or removed.

If not masked or removed, histocompatibility antigens that have an inhibitory effect may bind, for example, to KIR receptors (killer immunoglobulin-like receptors), NKG2 receptors, and LIL-R receptors (leukocyte immunoglobulin-like receptors) of immunocompetent cells. Examples of histocompatibility antigens with an inhibitory effect include, in particular, the embryonic groups HLA-E, HLA-F, and HLA-G.

The inhibitory effect is imparted by receptor-ligand bonds between the histocompatibility antigens (ligand) and receptors on the immunocompetent cells. By masking or removing the inhibitory part of the expressed histocompatibility antigens, their inhibitory effect on immunocompetent cells is prevented or at least reduced.

In addition, other histocompatibility antigens, in particular the classic HLA groups of Classes I and II (i.e., HLA-A to -C, or HLA-D), may also be masked or removed, thereby enhancing the immunological response to the vaccine to be prepared. However, it should be noted in this regard that some of the neoantigens require MHC-I (HLA groups A. B, and C), and some neoantigens require MHC-II (HLA groups DQB, DRB, etc.), and therefore, not all histocompatibility antigens should be removed or masked in all cells.

In the preparation 18 of cell membranes by lysing the cells, those cells on which a part of the expression pattern has been masked or removed are lysed. Cell membranes (or fragments of cell membranes) for injection are obtained in this manner. Lysis of the cells makes the cells unable to further divide and proliferate after injection and thereby trigger a substantial immunological response.

The steps of taking a tissue sample 12 and determining the expression pattern 14 serve to ascertain the individual communication structure between the malignant tumor and the immune system. In particular, this enables the identification of any escape mechanisms the malignant tumor uses to evade an immunological response.

The steps of masking or removal 16 and lysis 18 serve to prepare an individual vaccine for eliciting a specific immunological response. Preparation of the vaccine is carried out in vitro, in particular, i.e., prior to injection of the cell membranes. Based on the prior ascertainment of the individual communication structure between the malignant tumor and the immune system, the vaccine can also be individually produced.

Any neoantigens that are expressed as surface proteins by the extracted malignant tumor cells and are characteristic of the malignant tumor are not affected, or at least are not significantly affected, by the masking or removal of the inhibitory histocompatibility antigens, and therefore, they are also present on the prepared cell membranes. When the cell membranes with neoantigens are injected, the immune system responds to these neoantigens, in particular, and without inhibitory effect of the inhibitory histocompatibility antigens due to the masking, and initiates an immunological response.

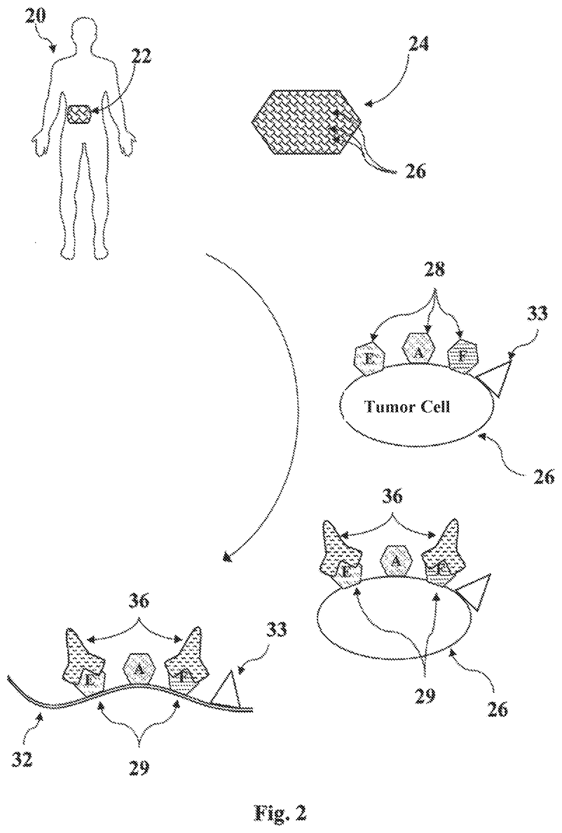

FIG. 2 is a schematic representation of a procedure for carrying out a method. Beginning at the top left and moving clockwise, a sequence of schematic views at different times during the procedure for carrying out the method are shown.

An individual 20 has a malignant tumor 22. In the case depicted, this is a primary tumor, although other exemplary embodiments involving metastases can also be implemented. Malignant tumor 22 may be malignant melanoma, for example. Malignant melanomas typically have a large number of neoantigens.

A tissue sample 24 comprising cells 26 of the malignant tumor 22 has been taken from the individual 20. On the tissue sample 24, in particular the extracted cells 26 of the malignant tumor 22, an expression pattern 28 of histocompatibility antigens is determined. In the case depicted here, this determination indicates that a cell 26 of the tissue sample 24 expresses the expression pattern 28 of three different groups of histocompatibility antigens, for example.

The three histocompatibility antigens in this case are proteins of the groups HLA-E, HLA-A, and HLA-F. The proteins of the HLA-E and HLA-F groups are capable of exerting an inhibitory effect on immunocompetent cells.

For example, HLA-F is capable of binding to LIL receptors of lymphocytes and diminishing the activity of the lymphocytes. Similarly, HLA-E is capable of binding to NKG2 receptors, for example, and diminishing the activity of natural killer cells. The groups HLA-E and HLA-F thus form a part 29 of the expression pattern 28 that can weaken or suppress the immunological response.

Inhibitory effect in this context refers to the immunomodulatory effect, which reduces or prevents the cytotoxic activity of immunocompetent cells. This signal pathway can be triggered, for example, via the immunoreceptor tyrosine-based inhibitory motif (ITIM), i.e., cytoplasmic phosphorylation.

Proteins of the HLA-A group are essentially incapable of exerting an inhibitory effect on immunocompetent cells.

In addition to the expression pattern 28, the cells 26 of the malignant tumor also include on their surface characteristic neoantigens 33 that are based on new mutations occurring during the course of malignant degeneration. The neoantigens 33 are depicted schematically here, although a determination of these neoantigens is not necessary.

In the next step, the part 29 of the expression pattern 28 that is capable of exerting an inhibitory effect on immunocompetent cells is masked by means of antibodies 36. Thus, the inhibitory part 29 in this case is the histocompatibility antigens of the groups HLA-E and HLA-F, but not HLA-A.

In other embodiments, classic HLA groups of Classes I or II (in this case HLA-A, for example) may also be masked, thereby enhancing the immunological response to the vaccine to be prepared. In the case depicted, HLA-A has not been masked because some neoantigens require MHC-I (HLA groups A, B, and C). Thus, in the exemplary embodiment shown, not all histocompatibility antigens have been masked in all cells, in order to ensure a specific activation of the immune system by the presented neoantigens.

The HLA-E groups can be masked by anti-HLA-E antibodies. The HLA-F groups can be masked by anti-HLA-F antibodies. In other exemplary embodiments, bivalent antibodies (anti-HLA-E/F) may alternatively be used. After being masked by the antibodies 36, the proteins of the HLA-E and HLA-F groups can no longer bind to the corresponding LIL and/or NKG2 receptors of immunocompetent cells, and thus exert no inhibitory effect on the immunocompetent cells.

In such a usage of antibodies to mask histocompatibility antigens that have an inhibitory effect, the binding of histocompatibility antigens to receptors present on immunocompetent cells is prevented or reduced. The antibodies preferably display a high affinity to the histocompatibility antigens, in particular comparable to, greater than, or substantially greater than the affinity of the immune receptors. The affinity is preferably great enough to prevent a diffusing off and/or a competitive displacement of the antibodies.

Once the immunoinhibitory part 29 of the expression pattern 28 has been masked, the cells 26 (on which a part of the expression pattern has been masked) are lysed to obtain cell membranes 32 for injection. In addition to the expression pattern 28 of histocompatibility antigens, the cell membranes 32 exhibit the neoantigens 33 characteristic of the malignant tumor, as well as the antibodies 36 that mask the inhibitory part 29 of the expression pattern 28.

Injection (not shown) of the cell membranes 32 may be performed into the individual 20 from whom the tissue sample 24 with malignant tumor cells 26 was taken.

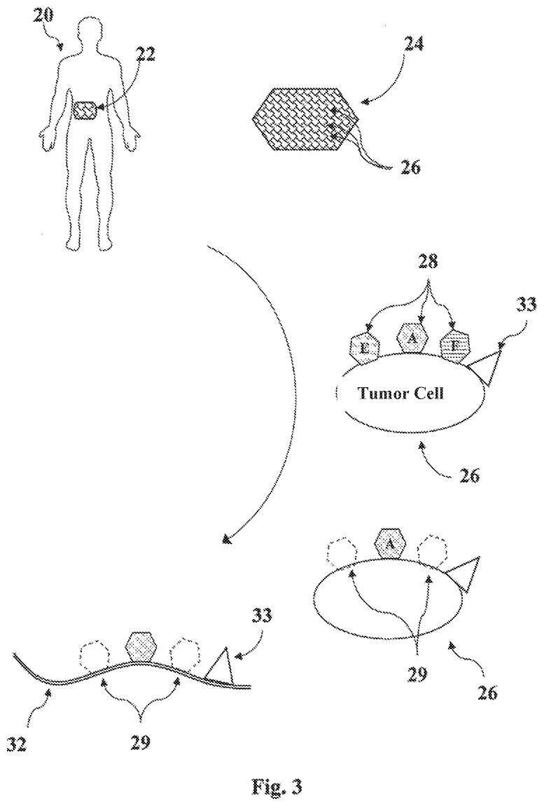

FIG. 3 shows a schematic view of a procedure for carrying out a further method. Beginning at the top left and moving clockwise, a sequence of schematic views at different times during the procedure for carrying out the method are shown.

In the method sequence depicted in FIG. 3, similarly to the method depicted in FIG. 2, a tissue sample 24 containing cells 26 of a malignant tumor 22 is taken from an individual 20, and the expression pattern 28 of histocompatibility antigens of at least one cell 26 is determined. The cells 26 have neoantigens 33 characteristic of the malignant tumor.

In contrast to the method illustrated in FIG. 2, however, according to FIG. 3, once the expression pattern has been determined, the part 29 of the expression pattern that is capable of exerting an inhibitory effect on immunocompetent cells is removed by means of gene manipulation techniques.

Similarly to the exemplary embodiment depicted in FIG. 2, HLA-A also has not been removed in the case shown here, since some neoantigens require MHC-I. Thus, in the exemplary embodiment shown, not all histocompatibility antigens are removed from all cells, in order to ensure a specific activation of the immune system by the presented neoantigens.

In the depicted case, this removal can be achieved by means of a Crispr/CAS procedure, in which the DNA segments coding for the histocompatibility antigens of groups HLA-E and HLA-F are excised from the genome of the cells 26, and the cells thus modified are cultured. Cells 26 are thereby prepared that are substantially identical to the extracted malignant tumor cells, but do not express the immuno-inhibitory part 29 of the histocompatibility antigens (indicated schematically by dashed outlines of the part 29). In particular, the cells at least express the neoantigens 33 characteristic of the malignant tumor.

Those cells 26 from which the inhibitory part 29 has been removed are lysed to obtain cell membranes for injection. Due to the removal of the part 29, the cell membranes 29 exert no inhibitory effect, or at least a reduced inhibitory effect, on the immune system. However, they still include neoantigens 33 that enable them to trigger an immunological response after injection.

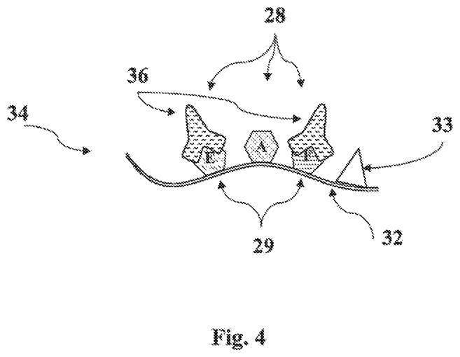

FIG. 4 shows a schematic view of a vaccine 34 from a cell membrane 32, prepared from a malignant tumor (not shown), by a method according to the invention. This may be a cell membrane prepared according to the exemplary embodiment of FIG. 2, for example.

The cell membrane 32 has an expression pattern 28 of histocompatibility antigens. A part 29 of the expression pattern 28 that is capable of inhibiting an inhibitory response of the immune system has been masked by antibodies 36. The inhibitory effect of the part 29 of the expression pattern 28 on the immune system can thereby be prevented or at least reduced.

In addition to the expression pattern 28, the cell membrane 32 further has neoantigens 33. The neoantigens are proteins based on new mutations of the genome of malignant tumor cells occurring during the course of malignant degeneration. The neoantigens are characteristic of the malignant tumor and are capable of eliciting an immune system response (if the immunological response is not inhibited).

The depicted cell membrane 32 is suitable for use as a vaccine 34 for treating a malignant tumor by the specific activation of the immune system.

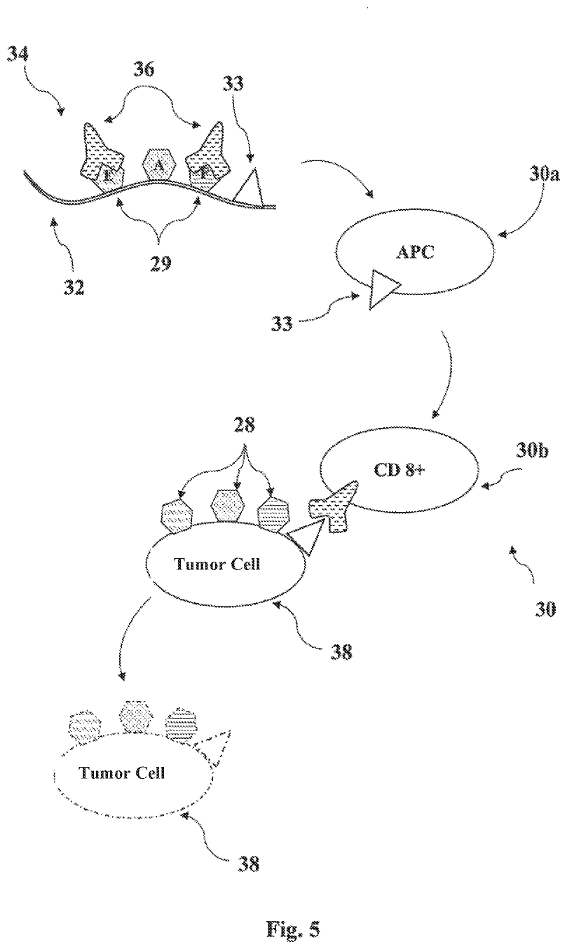

FIG. 5 shows a schematic representation of a use of a cell membrane 32 according to FIG. 4 as a vaccine 34 for treating a malignant tumor. The cell membrane 32 has been prepared from a tissue sample containing cells of the malignant tumor to be treated.

For use as a vaccine, the cell membrane 32 with bound antibodies 36 that mask an inhibitory part 29 of the expression pattern of histocompatibility antigens, and with neoantigens 33, is injected into the organism afflicted by the malignant tumor.

The immunocompetent cells of the organism recognize some of the proteins presented by the cell membrane 32 as foreign antigens. If neoantigens 33 not recognized by the organism are expressed and presented on the cell membranes, a corresponding immunological response, e.g., the formation of antibodies and/or the activation of T cells, will develop. Since no immunoinhibitory histocompatibility antigens are “visible”, this immunological response will not be blocked.

In the case depicted here, the cell membranes 32 prepared according to the invention exhibit, in particular, two interactions with the immune system 30: Firstly, the neoantigens 33, which are characteristic of the malignant tumor, elicit an adaptive immunological response to these malignant tumor-characteristic neoantigens.

Secondly, because the part 29 of the histocompatibility antigens has been masked by antibodies 36, its inhibitory effect on the immunological response is prevented or at least reduced. In the unmasked state, part 29 would be capable of inhibiting a response by the immune system 30, in particular to the neoantigens 33.

The immune system 30 of the organism triggers an immunological response. The immune system 30, in the schematic representation, comprises immunocompetent cells 30a, 30b, specifically, antigen-presenting cells (APC) 30a and CD8+ T cells 30b.

Based on the adaptive immunological response (depicted here schematically in the form of antigen-presenting cells 30a and T cells 30b), the immune system 30 is able to recognize any cells 38 having the underlying neoantigens 33. These include, in particular, the cells of the malignant tumor from the tissue sample of which the vaccine in the form of the cell membrane 32 was obtained. By preparing the vaccine individually, the immunological response can be directed toward those neoantigens 33 that are actually expressed in the malignant tumor, without having to first determine the new mutations or neoantigens by elaborate sequencing, for example.

The immunological response may be implemented by CD8+ T cells 30b with T-cell receptors, for example. The cytotoxic T cells 30b may have been activated by antigen-presenting cells 30a, which present the neoantigen 33 or a partial peptide of the neoantigen 33. When the activated T cells 30b recognize a malignant tumor cell 38 based upon the neoantigen 33, they initiate apoptosis of the malignant tumor cells (represented by dashed outlines of the malignant tumor cell 38).

1. A method for preparing a medicament for treating an individual with a malignant tumor, said method comprising:
(a) ascertaining the individual communication structure between the malignant tumor and the immune system, which involves
determining a malignant tumor-specific expression pattern of histocompatibility antigens (Human Leucocyte Antigen, HLA) on a tissue sample that contains cells of the malignant tumor,
(b) preparing an individual vaccine to elicit a specific immunological response, which involves
masking or removing at least one part of the expression pattern, present on the cells of the tissue sample, that is capable of exerting an inhibitory effect on immunocompetent cells, and
lysing those cells on which a part of the expression pattern has been masked or removed, to thereby obtain cell membranes or fragments of cell membranes for an injection.

2. The method according to claim 1, in which the histocompatibility antigens for which the expression pattern is determined comprise embryonic HLA groups, in particular HLA-E, F, and/or G.

3. The method according to claim 2, in which the part of the expression pattern to be masked or removed comprises the embryonic HLA groups, in particular HLA-E, F, and/or G.

4. The method according to any one of the preceding claims, in which the at least one part of the expression pattern is masked by means of antibodies.

5. The method according to any one of the preceding claims, wherein the at least one part of the expression pattern is removed by means of gene manipulation techniques.

6. The method according to any one of the preceding claims, in which the cells are lysed by means of mechanical or biochemical cell disruption methods, in particular by means of hypotonic lysis.

7. The method according to any one of the preceding claims, in which the cell complex of the tissue sample is dissociated to obtain a single cell suspension.

8. Cell membranes prepared by a method according to any one of the preceding claims.

9. A use of cell membranes according to claim 8 as a medicament for treating a malignant tumor, in particular as a vaccine for the specific activation of the immune system.

10. The use according to claim 9 as a vaccine containing cell membranes or at least fragments of said cell membranes for the organism from which the tissue sample was taken, in order to elicit a specific activation or response of the immune system.
